(19) United States
(12) Patent Application Publication
(10) Pub. No.: US 2010/0092980 A1
(43) Pub. Date: Apr. 15, 2010

(54) REAGENT FOR ELIMINATION OF RED BLOOD CELLS AND HEMOGLOBIN IN A SAMPLE

(75) Inventors: Tzong-Hae Lee, San Francisco, CA (US); Leilani Montalvo, Hayward, CA (US); Li Wen, Albany, CA (US)

Correspondence Address: DLA PIPER LLP (US), 4365 EXECUTIVE DRIVE, SUITE 1100, SAN DIEGO, CA, 92121-2133, US

(73) Assignee: Blood Systems, Inc., Scottsdale, AZ (US)

(21) Appl. No.: 12/572,073

(22) Filed: Oct. 1, 2009

Related U.S. Application Data
(60) Provisional application No. 61/102,302, filed on Oct. 2, 2008.

(51) Int. Cl. C12Q 1/68

(52) U.S. Cl. 435/6

(57) ABSTRACT

The invention relates to a method for isolating nucleic acid from a blood sample, including individual blood samples or pooled samples, such as in blood banks. Furthermore, this invention relates to a reagent kit suitable for carrying out the method of the invention.

### RELATED APPLICATIONS

This application is a utility application and claims priority under 35 U.S.C. §119(e) of U.S. Provisional Application Ser. No. 61/102,302 filed Oct. 2, 2008, the entire content of which is incorporated herein by reference.

### BACKGROUND OF THE INVENTION

1. Field of the Invention

The invention relates generally to a method for removing red blood cells and hemoglobin from a sample and, more specifically, to a reagent for use with blood samples to allow increased sensitivity in nucleic acid detection.

2. Background Information

Blood from blood donations is used for transfusions or made into medications. Blood donors are screened for health risks that might make the donation unsafe for the recipient. Screening includes examination for signs and symptoms of diseases that can be transmitted in a blood transfusion, such as Human Immunodeficiency Virus (HIV), malaria, and viral hepatitis. Screening may also include questions about risk factors for various diseases, such as travel to countries at risk for malaria or variant Creutzfeldt-Jakob Disease (vCJD). Donated blood is also tested for the presence of a transfusion transmitted infection (TTI), including infection by a virus, parasite, or any other potential pathogen that can be transmitted in blood. The term is usually limited to known pathogens, but also sometimes includes agents such as Simian foamy virus which are not known to cause disease.

Preventing the spread of TTIs by blood transfusion is addressed in several ways. In many cases, the blood is tested for the pathogen, sometimes with several different methodologies. The core tests recommended by the World Health Organization (WHO) are: Hepatitis B Surface Antigen, antibody to Hepatitis C, antibody to HIV (usually subtypes 1 and 2) and a serologic test for Syphilis. However, the WHO reported in 2006 that 56 out of 124 countries surveyed did not use these basic tests on all blood donations. A variety of other tests for TTIs are often used based on local requirements. However, additional testing is expensive, and in some cases the tests are not implemented because of the cost.

The most common diseases transmitted in blood transfusions are viral infections. Current laboratory screening tests fall into three categories: antibody tests, nucleic acid tests (NAT), and surrogate tests. Antibody tests assay for the immune system's response to the infection. Nucleic acid tests look for the genetic material of the TTI itself. The third category are tests that are not specific to the disease but look for other related conditions.

Methods for recovering nucleic acids for use in NAT include the steps of: isolating the nucleic acids from cells and tissues by lysing the materials under highly denaturing and reducing conditions, partly using protein-degrading enzymes, purifying the nucleic acid fractions obtained by means of phenol/chloroform extraction processes and recovering the nucleic acids from the aqueous phase by dialysis or ethanol precipitation. There are also a number of commercially available reagent systems for purifying DNA fragments and also for isolating longer-chained nucleic acids (genomic DNA, total cell RNA) from blood, tissues or cell cultures. Many of these commercially available purification systems use mineral carriers to bind DNA in the presence of solutions of different chaotropic salts. In these systems, suspensions of finely ground glass powder, diatomaceous earth or silica gels are used as carrier materials.

However, a major disadvantage of the currently available processes is that these processes do not include removal of red blood cells (RBCs) and hemoglobin from the sample. Furthermore, the procedures are not suitable for all materials and/or are extremely inefficient for small quantities of starting materials. NATs can detect TTIs classically considered to exist exclusively in the plasma compartment of blood (e.g., HIV, HBV, HCV, WNV). However, NAT donor blood is not routinely screened for the presence of several TTIs that are associated with cells, such as parvovirus B19, Dengue virus, or ChikV.

Substantial proportions of TTI nucleic acids in blood are in or bound to platelets, red blood cells (RBCs) or white blood cells (WBCs), and thus are not detected by plasma or serum analysis. In addition, there are a number of primarily cell-associated transfusion transmitted viruses (e.g., CMV, EBV, and HHV-8) and parasites (e.g., T cruzi, Plasmodium, Borrelia, Leishmania, and Babesia) for which large scale donor screening is not currently possible due to the inability of high-throughput, automated sample processing systems to accept whole blood as input specimens. Further, detection of early phase and so-called “occult” infections which are associated with very low levels of plasma RNA/DNA are not detected using current plasma screening techniques.

### SUMMARY OF THE INVENTION

The present invention describes a method for detecting nucleic acid in a sample by removing RBCs and hemoglobin from the sample, e.g., whole blood. Additionally, the invention describes a method for capturing a nucleic acid from whole blood. The invention offers several advantages over current NATs in that the method can be used to isolate nucleic acids from TTIs that are associated with cells; to isolate TTIs that are present in low copy number; and to isolate TTI nucleic acid in whole blood.

The present invention relates to a method for isolating nucleic acid from whole blood by removing or releasing nucleic acid from RBCs or hemoglobin to which it might be associated, as well as lysing cells in the whole blood that may contain or have bound thereto microbial nucleic acid, for example. The treated or lysed sample is contacted with a reagent of the invention that binds the RBCs and hemoglobin in blood to make the nucleic acid more accessible to form a lysed cells/reagent sample. The lysed cells/reagent sample contains particles of reagent bound to lysed or intact blood cells (e.g., RBCs, WBCs, or platelets), blood cell debris, and hemoglobin. The invention reagent comprises a polymer, such as polyethylene glycol (PEG) and a salt. In one embodiment of the invention, the polyethylene glycol is PEG 8000. In another embodiment, the polyethylene glycol is present in an amount of about 20 to 40% of the reagent volume. In one embodiment, the salt is a chloride salt, such as sodium chloride, potassium chloride, calcium chloride, ammonium chloride, iron chloride and the like. In one embodiment, the salt is present in the reagent in a concentration range of about 2.5-5.0 moles per liter. In one particular embodiment, the cells in the blood sample are lysed by contact with an agent effective in inactivating nucleases and releasing nucleic acid particles, such as a strong chaotropic agent including guanidine salts, for example, guanidinium chloride. In one aspect, the guanidine salt is guanidine-HCl. As a buffered reagent, guanidine-HCl-EDTA is used for example. Other chaotropic agents are known to the skilled artisan and include urea and lithium perchlorate.

In one embodiment, the RBCs and hemoglobin are removed from the lysed cells/reagent sample by centrifugation. In another embodiment, the RBCs and hemoglobin are removed from the lysed cells/reagent sample by filtration. In yet another embodiment, the RBCs and hemoglobin are removed from the lysed cells/reagent sample by settling. Other commonly known techniques can be used to separate the RBCs/hemoglobin/lysed cells/reagent from the supernatant containing nucleic acid.

The methods described herein are useful for detecting nucleic acid from an infectious agent in a blood sample. The sample can be from an individual or it can be from a pooled sample of blood (e.g., a blood bank). In one embodiment, the infectious disease is caused by a microorganism. The microorganism can be a virus, a bacteria, or a parasite. The virus diagnosed can be HIV, hepatitis A virus, HBV, HCV, WNV, Parvovirus B19, HTLV I/II, simian foamy virus, SARS, Dengue, ChikV, CMV, EBV, and HHV-8. The bacteria diagnosed bacteria can be Escherichia, Proteus, Klebsiella, Staphylococcus, Streptococcus, Pseudomonas and Lactobacillus. The parasite diagnosed can be Plasmodium, Leishmania, Babesia, Treponema, Borrelia and Trypanosoma.

Accordingly, one aspect of the invention provides a method for capturing a nucleic acid from whole blood by lysing cells in a sample, contacting the lysed cell sample with a reagent comprising polyethylene glycol (PEG) and a salt, clarifying the lysed cells/reagent to remove RBCs and hemoglobin and lysed cell debris, and capturing the nucleic acid from the sample.

In one embodiment of the invention, the isolated nucleic acid is DNA or RNA. In one embodiment, the DNA or RNA is viral, prokaryotic, or eukaryotic in origin. In another aspect, the viral nucleic acid is derived from a virus selected from the group consisting of HIV, hepatitis A virus, HBV, HCV, WNV, Parvovirus B19, HTLV I/II, simian foamy virus, SARS, Dengue, ChikV, CMV, EBV, and HHV-8. In one embodiment, the prokaryotic DNA is derived from a prokaryote selected from the group consisting of Escherichia, Proteus, Klebsiella, Staphylococcus, Streptococcus, Pseudomonas and Lactobacillus. In another embodiment, the eukaryotic DNA is derived from a eukaryote selected from the group consisting of algae, protozoa, parasites, fungi, molds and mammalian cells. In yet another embodiment, the parasitic DNA is derived from a parasite selected from the group consisting of Plasmodium, Leishmania, Babesia, Treponema, Borrelia and Trypanosoma.

In one embodiment, the methods of the invention further include diagnosing infectious disease by analyzing the captured nucleic acid. In another embodiment, the methods of the invention include diagnosing a subject as having or at risk of having a transfusion transmitted infection (TTI) by detecting the presence of TTI nucleic acids in a test sample from the subject, wherein detecting the presence of TTI nucleic acids is diagnostic of a TTI. In one embodiment, the TTI disease is caused by a microorganism. In another aspect, the microorganism is a virus, a bacteria, or a parasite.

In a further embodiment of the invention, there are provided methods of preparing a blood sample for nucleic acid analysis. The method includes contacting a blood sample with a lysis reagent to form a lysed blood sample, contacting the lysed blood sample with a reagent that binds to blood cells and hemoglobin to form particles of reagent bound to the blood cells and hemoglobin; and separating the particles from the supernatant, wherein the nucleic acids remain in the supernatant, thereby preparing the sample for nucleic acid analysis. In some embodiments, the separating step includes settling, filtering, or centrifuging. The method may further isolating the supernatant and, optionally, additional steps to extract or purify the nucleic acids in the supernatant. In particular embodiments, the lysis reagent comprises a chaotropic agent, such as guanidinium chloride, guanidine HCl, urea, or lithium perchlorate. In one aspect, the chaotropic agent is guanidine HCl. In particular embodiments, the reagent that binds to blood cells and hemoglobin comprises polyethylene glycol. In one aspect, the polyethylene glycol is present in an amount of about 20 to about 40% of the reagent volume. The reagent that binds to blood cells and hemoglobin may further comprise a salt. In a particular aspect, the salt is present in a concentration of about 2.5 M to about 5 M.

In another embodiment, a kit for detecting TTI in a blood sample is provided. The kit includes a reagent for removing RBCs and hemoglobin from whole blood, such as a reagent comprising polyethylene glycol (PEG) and a salt, such as NaCl. In one aspect, the kit also contains a lysis buffer comprising guanidine-HCl.

### BRIEF DESCRIPTION OF THE DRAWINGS

FIG. 1 is a flow chart depicting invention methods for nucleic acid isolation from whole blood.

FIG. 2 is a gel depicting Trypanosoma cruzi nucleic acid isolated from whole blood and amplified by nested PCR.

FIG. 3 is a graph depicting Parvovirus B19 nucleic acid samples isolated from whole blood or plasma followed by amplification using TC-RT or ultracentrifuge RT.

FIG. 4A is a graph depicting the viral load of Parvovirus B19 in clinical donor specimens using nucleic acid samples isolated from whole blood or plasma. FIG. 4B is a graph depicting the correlation of Parvovirus B19 nucleic acid recovery from whole blood verses plasma.

### DETAILED DESCRIPTION OF THE INVENTION

The present invention is based on a method for detection of nucleic acid in a blood sample. The method includes removing RBCs and hemoglobin as well as cellular material from whole blood. The method of the invention includes lysing cells in whole blood prior to removal, leaving nucleic acid intact for detection. Cells in the sample may be lysed with solutions containing, for example, guanidine, guanidine HCl, guanine thiocyanate or certain other chaotropic agents and detergents.

The invention also provides a method for capturing a nucleic acid from whole blood. Blood cells and hemoglobin are removed from a sample by lysing the cells with a chaotropic agent and contacting the lysed sample with a reagent that binds to the lysed cells and hemoglobin to form particles, wherein the nucleic acid remains in the supernatant. The nucleic acids in the supernatant may be captured by methods known in the art (e.g., magnetic beads having oligonucleotides designed to capture nucleic acids of interest). The captured nucleic acids can then be analyzed using standard protocols that are well known in the art, such as PCR. As such, the methods of the invention allow for rapid screening of TTIs in whole blood specimens to enable screening of donor blood for TTIs, as well as screening for infectious agents other than those for which there is significant plasma viremia.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The reagent that binds to blood cells and hemoglobin used in the methods of invention includes polyethylene glycol (PEG) and salt. PEGs are commercially available over a wide range of molecular weights from about 300 g/mol to about 10,000,000 g/mol. Thus, in one embodiment, the PEG used in the reagent has a molecular weight ranging from about 300 g/mol to 10,000,000 g/mol. In another embodiment, the molecular weight of the PEG is 8000 g/mol (PEG 8000). The PEG can be present in the reagent in varying amounts, for example, in an amount of about 20 to 40% of the reagent volume. In one embodiment, the salt is a halide. In another embodiment, the salt a chloride, such as sodium chloride, potassium chloride, calcium chloride, ammonium chloride, iron chloride and the like. In one embodiment, the salt is present in the reagent in a concentration range of about 2.5-5.0 moles per liter.

The phrase “nucleic acid” means DNA, RNA, single-stranded, double-stranded or triple stranded and any chemical modifications thereof A “nucleic acid” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule. The nucleic acid sample of interest will be one which is suspected of containing a particular target nucleic acid, such as a particular gene, gene segment or RNA. In one embodiment, the nucleic acid is derived from eukaryotes, prokaryotes, viruses or parasites.

As used herein, the terms “sample” and “biological sample” refer to any sample suitable for the methods provided by the present invention. The sample can be any sample, including, for example, a sample containing a wide range of eukaryotic and prokaryotic cells, or a sample of the subject's body fluid. Thus, in one embodiment, the sample of the present invention is a biological sample, e.g., blood, serum, or plasma. In one aspect, the blood sample is a pooled blood sample, such as in a blood bank.

In one embodiment, the methods of the present invention are useful, for instance, for detecting and diagnosing non-pathogenic or pathogenic microorganisms of interest. Exemplary microorganisms include, but are not limited to, viruses, bacteria, or parasites. The presence and identity of the microorganisms may be established, by detecting homology between the sequences of nucleic acids of a known source and nucleic acids resident in the sample. For example, the invention may assist in the diagnosis of various infectious diseases by isolation of particular nucleic acid sequences known to be associated with a particular microorganism. Additionally, since the methods of the invention isolate low copy nucleic acid, very low-level carriers of HIV (“elite controllers”) and HBV (“occult HBV” infections) can be identified.

Thus, in one embodiment, the methods of the invention isolate nucleic acid. The nucleic acid can be viral, prokaryotic, or eukaryotic in origin. Exemplary viruses from which nucleic acid is derived include, but are not limited to, HIV, hepatitis A virus, HBV, HCV, WNV, Parvovirus B19, HTLV I/II, simian foamy virus, SARS, Dengue, ChikV, CMV, EBV, and HHV-8. Exemplary prokaryotes from which nucleic acid is derived include, but are not limited to, Escherichia, Proteus, Klebsiella, Staphylococcus, Streptococcus, Pseudomonas and Lactobacillus. Exemplary eukaryotes from which nucleic acid is derived include, but are not limited to, algae, protozoa, parasites, fungi, molds and mammalian cells. Exemplary parasites from which nucleic acid is derived include but are not limited to Plasmodium, Leishmania, Babesia, Treponema, Borrelia and Trypanosoma.

After isolation, the nucleic acids can be further manipulated using assays that are well known in the art. For example, synthetic oligonucleotide primers can be used to amplify an RNA or DNA template (see U.S. Pat. Nos. 4,683,195 and 4,683,202; PCR Protocols: A Guide to Methods and Applications (Innis et al., eds, 1990), incorporated herein by reference). Methods such as polymerase chain reaction (PCR and RT-PCR) and ligase chain reaction (LCR) can be used to amplify nucleic acid sequences directly from RNA and DNA. PCR or other in vitro amplification methods may also be useful, for example, to clone nucleic acid sequences, for other purposes, such as, for example, nucleic acid sequencing.

Additionally, sandwich assays are commercially useful for detecting or isolating nucleic acid sequences. Such assays utilize a “capture” nucleic acid covalently immobilized to a solid support and labeled “signal” nucleic acid in solution. In one embodiment, the isolated nucleic acid is the target nucleic acid. As such, the “capture” nucleic acid and “signal” nucleic acid probe hybridize with the target nucleic acid to form a “sandwich” hybridization complex. To be effective, the signal nucleic acid is designed so that it cannot hybridize with the capture nucleic acid, but will hybridize with the target nucleic acid in a different position than the capture probe.

Virtually any solid surface can be used as a support for hybridization assays, including metals and plastics. Different types of solid surfaces are commercially available, including membranes, polystyrene beads, nylon, Teflon, polystyrene/latex beads, latex beads or any solid support possessing an activated carboxylate, sulfonate, phosphate or similar activatable group are suitable for use as solid surface substratum to which nucleic acids or oligonucleotides can be immobilized. Additionally, porous membranes possessing pre-activated surfaces which may be obtained commercially (e.g., Pall Immunodyne Immunoaffinity Membrane, Pall BioSupport Division, East Hills, N.Y., or Immobilon Affinity membranes from Millipore, Bedford, Mass.) and which may be used to immobilize capture oligonucleotides. Finally, microbeads, including magnetic beads, of polystyrene, teflon, nylon, silica or latex may also be used.

The nucleic acids isolated by the methods of the invention can be identified using stringent hybridization conditions, or conditions under which a probe will hybridize to its target subsequence, typically in a complex mixture of nucleic acids, but to no other sequences. Typically, high stringency conditions are desirable in order to prevent or at least minimize false positives. Stringent conditions are sequence-dependent and will be different in different circumstances. For example, longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions include, but are not limited to, 50% formamide, 5×SSC, and 1% SDS, incubating at 42° C., or, 5×SSC, 1% SDS, incubating at 65° C., with wash in 0.2×SSC, and 0.1% SDS at 65° C.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.

Real-time PCR, also known as quantitative real time polymerase chain reaction (qPCR) or kinetic polymerase chain reaction, can be used to simultaneously amplify and quantify a target DNA molecule. It enables both detection and quantification (as absolute number of copies or relative amount when normalized to DNA input or additional normalizing genes) of a specific sequence in a DNA sample.

Sequences suitable for capture or signal nucleic acids for use in hybridization assays can be obtained from the entire sequence or portions thereof of an organism's genome, from messenger RNA, or from cDNA obtained by reverse transcription of messenger RNA. Methods for obtaining the nucleotide sequence from such obtained sequences are well known in the art (see Ausubel et. al in Current Protocols in Molecular Biology, pub. John Wiley & Sons (1998), and Sambrook et al. in Molecular Cloning, A Laboratory Manual, Cold Spring Habor Laboratory Press, 1989). Furthermore, a number of both public and commercial sequence databases are accessible and can be used to obtain the relevant sequences.

Other techniques can be used to identify known and previously uncharacterized nucleic acid sequences isolated by the method of the invention, including representational difference analysis (RDA), DNA microarrays and use of degenerate PCR primers or other methods well known to those of skill in the art.

When synthesizing a primer or probe for a specific target, the choice of nucleotide sequence will determine the specificity of the test. For example, by comparing DNA sequences from several virus isolates, one can select a sequence for virus detection that is either type specific or genus specific. Comparisons of DNA regions and sequences can be achieved using commercially available computer programs.

Primers and probes can additionally be labeled in various ways depending on the choice of label. Radioactive probes are typically made by using commercially available nucleotides containing the desired radioactive isotope. The radioactive nucleotides can be incorporated into probes by several means such as by nick translation of double-stranded probes; by copying single-stranded M13 plasmids having specific inserts with the Klenow fragment of DNA polymerase in the presence of radioactive dNTP; by transcribing cDNA from RNA templates using reverse transcriptase in the presence of radioactive dNTP; by transcribing RNA from vectors containing SP6 promoters or T7 promoters using SP6 or T7 RNA polymerase in the presence of radioactive rNTP; by tailing the 3′ ends of probes with radioactive nucleotides using terminal transferase; or by phosphorylation of the 5′ ends of probes using [₃₂P]-ATP and polynucleotide kinase.

Non-radioactive probes are often labeled by indirect means. Generally, a ligand molecule is covalently bound to the probe. The ligand then binds to an anti-ligand molecule which is either inherently detectable or covalently bound to a signal system, such as a detectable enzyme, a fluorescent compound, or a chemiluminescent compound. Ligands and anti-ligands may be varied widely. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The probe may be conjugated directly with the label. For example, where the label is radioactive, the probe with associated hybridization complex substrate is exposed to X-ray film. Where the label is fluorescent, the sample is detected by first irradiating it with light of a particular wavelength. The sample absorbs this light and then emits light of a different wavelength which is picked up by a detector (Physical Biochemistry, Freifelder, D., W. H. Freeman & Co. (1982), pp. 537-542). Where the label is an enzyme, the sample is detected by incubation on an appropriate substrate for the enzyme. The signal generated may be a coloured precipitate, a colored or fluorescent soluble material, or photons generated by bioluminescence or chemiluminescence.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label. (Tijssen, P., “Practice and Theory of Enzyme Immunoassays,” Laboratory Techniques in Biochemistry and Molecular Biology, Burdon, R. H., van Knippenberg, P. H., Eds., Elsevier (1985), pp. 9-20.)

The nucleic acids isolated by the methods of the present invention can be used for detection of a pathogen, diagnosis of a pathogen, and determination of the quantity of pathogen present such as by determination of viral load. Accordingly, in another aspect, the present invention provides methods of diagnosing a subject as having or at risk of having a TTI. The method includes comparing TTI nucleic acid activity or expression in a test sample from the subject with TTI nucleic acid activity or expression in a corresponding normal sample. An altered level of TTI nucleic acid activity or expression in the test sample as compared to the TTI nucleic acid activity or expression in the corresponding normal sample is indicative of the presence of a TTI in the subject.

In another embodiment, the methods of the invention are useful for providing a means for practicing personalized medicine, wherein treatment is tailored to a subject based on the particular characteristics of the TTI in the subject. The method can be practiced, for example, by first diagnosing the TTI, as described above.

The sample of cells examined according to the present method can be obtained from the subject to be treated, or can be cells of an established cell line of the same type as that of the subject. In one aspect, the established cell line can be one of a panel of such cell lines, wherein the panel can include different cell lines of the same type of disease and/or different cell lines that are capable of supporting TTI infection. Such a panel of cell lines can be useful, for example, to practice the present method when only a small number of cells can be obtained from the subject to be treated, thus providing a surrogate sample of the subject's cells, and also can be useful to include as control samples in practicing the present methods.

Once disease is established and a treatment protocol is initiated, the methods of the invention may be repeated on a regular basis to monitor the TTI nucleic acid activity or expression level in the subject. The results obtained from successive assays may be used to show the efficacy of treatment over a period ranging from several days to months. Accordingly, another aspect of the invention is directed to methods for monitoring a therapeutic regimen for treating a subject having a TTI. A comparison of the expression level or activity of the TTI nucleic acid prior to and during therapy will be indicative of the efficacy of the therapy. Therefore, one skilled in the art will be able to recognize and adjust the therapeutic approach as needed.

The efficacy of a therapeutic method over time can be identified by an absence of symptoms or clinical signs of the TTI in a subject at the time of onset of therapy. In subjects diagnosed as having the TTI disorder, the efficacy of a therapeutic method can be evaluated by measuring a lessening in the severity of the signs or symptoms in the subject or by the occurrence of a surrogate end-point for the disorder.

In another embodiment, the invention provides kits for detection of nucleic acid including removal of RBCs and hemoglobin from whole blood, and kits for the capture of nucleic acids. Such kits contain at least one vial containing a reagent comprising polyethylene glycol and salt. In one embodiment, the kit further contains a chaotropic salt and nuclease inhibitor, such as a lysis buffer containing guanidine-HCl EDTA.

The following examples are intended to illustrate but not limit the invention.

### Example 1

### Protocol for Targeted Nucleic Acid Capture from Whole Blood

This example demonstrates a method of the invention for the capture of nucleic acids from whole blood.

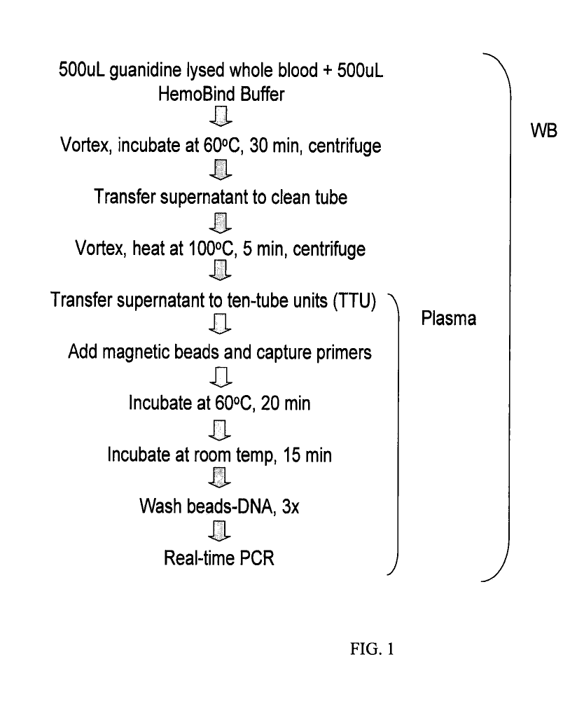

FIG. 1 shows a protocol for capturing nucleic acid from whole blood. The reagent (“HemoBind buffer”) is added in an equal volume to the whole blood after the whole blood is treated with guanidine-HCL EDTA lysis buffer. The lysis buffer inactivates nucleases and releases nucleic acid particles. The lysed cell plus reagent are vortexed and then incubated at 60° C. for thirty minutes. The cell plus reagent is then centrifuged to produce a supernatant. The supernatant is removed, vortexed, incubated at 100° C. for five minutes, and then centrifuged to produce a clarified supernatant. The clarified supernatant is transferred to another vial. Magnetic beads and capture primers are added to the clarified supernatant, followed by incubation at 60° C. for twenty minutes and a further incubation at room temperature for fifteen minutes. The magnetic beads are then washed, and real-time PCR is performed on the resulting nucleic acids.

### Example 2

### Isolation of Trypanosoma cruzi Nucleic Acids from Whole Blood

This example demonstrates that nucleic acids from Trypanosoma cruzi (Chagas disease) can be isolated from whole blood using the method of the invention.

Spiking Samples

Epimastigotes of Trypanosoma cruzi (Chagas disease), grown in an anexic culture, were harvested, counted, and spiked into fresh whole blood to create samples containing 128, 64, 32, 16, 8, 4, 2, and 1 parasite per 20 ml of whole blood. A further two-fold dilution of 1 parasite was created to ensure that the end point was reached (0 parasite).

The spiked parasitic samples were tested using one of three protocols. The first amplification protocol utilized a nested PCR protocol (“Nested PCR” in Table 1 below). The second protocol utilized the protocol described in Example 1 (“Real-time PCR” in Table 1 below). The third protocol utilized Qiagen-RTPCR (“Qiagen RTPCR” in Table 1 below).

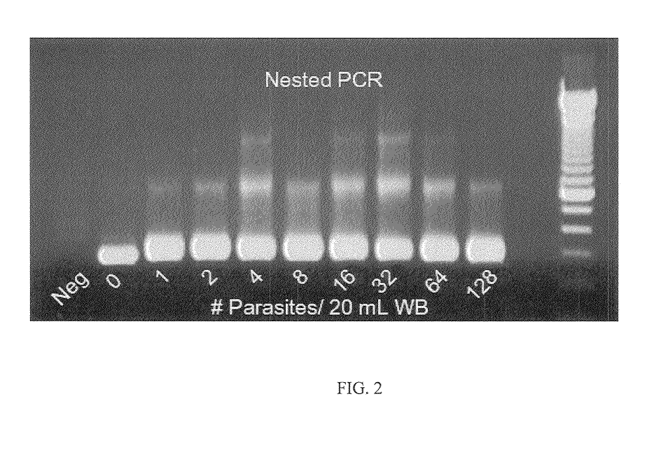

Table 1 lists the results from the three amplification protocols. The results of the second and third protocols are reported in Cycle Thresholds. The results from the nested PCR are also shown in FIG. 2.

TABLE 1

| | Method | | |
|---|---|---|---|
| | | Real-time | Qiagen- |
| Number of parasites | Nested | PCR | RTPCR |
| per 20 mL whole blood | PCR | n = 5 | n = 15 |
| 128 | Positive | Not tested | 29.1 |
| 64 | Positive | Not tested | 31.7 |
| 32 | Positive | Not tested | 32 |
| 16 | Positive | Not tested | 34.1 |
| 8 | Positive | 31.4 (+/−0.5) | 36.5 |
| 4 | Positive | 32.64 (+/−0.1) | 34.2 |
| 2 | Positive | 33.48 (+/−0.1) | 37.6 |
| 1 | Positive | 35.18 (+/−0.4) | 39.2 |
| 0 | Positive | 38.21 (+/−0.6) | Negative |
| Negative control | Negative | >45/>45 | Not reported |

Clinical Donor Specimens

Twenty-seven specimens were collected from donors who tested positive for T. cruzi by ELISA serology. Seven out of the twenty-seven samples were confirmed to be T. cruzi antibody positive by RIPA confirmatory testing. A panel of eighty-six American Red Cross donor specimens, including 72 RIPA positive and 14 RIPA negative donors were tested using the protocol described in Example 1. As shown in Table 2, real-time PCR using parasitic nucleic acids isolated with the method of the invention detected T. cruzi DNA in 13/79 seropositive donors.

TABLE 2

RIPA

| n = 113 | | Positive | Negative |
|---|---|---|---|
| PCR | Positive | 13 | 1* |
| | Negative | 66 | 33 |

*C_{T} = Neg, Neg, 39, 42 (PCR indeterminate)

These results demonstrate that nucleic acids from Trypanosoma cruzi (Chagas disease) can be isolated from whole blood using the method of the invention.

### Example 3

### Isolation of Parvovirus B19 Nucleic Acids from Whole Blood

This example demonstrates that nucleic acids from Parvovirus B19 can be isolated from whole blood using the method of the invention.

Spiking Samples

Spiking samples were prepared using CBER positive controls. The CBER control was spiked into Parvovirus B-19 negative plasma and Parvovirus B-19 negative whole blood to generate plasma and whole blood with the following serially diluted viral concentrations: 1000, 500, 250, 125, 62.5, 31.25 IU/mL.

Nucleic acids were isolated from the plasma or whole blood as described below.

TC-RT protocol: Four plasma samples (“TC-RT Plasma”) were processed. Nucleic acids from four whole blood samples (“TC-RT WB”) were isolated using the method of the invention, followed by target capture and real-time PCR (i.e., the protocol of Example 1).

Ultracentrifuge protocol: Four plasma samples (“Ultracentrifuge-RT plasma”) were ultracentrifuged for 2 hours (4° C.) at 50,377×g (Sovall Stratos) to pellet viral particles. The supernatant was removed and the pellet digested overnight with proteinase K. Four whole blood samples (“Ultracentrifuge-RT WB”) were subjected to RBC lysis to remove as many red blood cells possible, followed by ultracentrifugation to pellet viral particles. Viral particles from all samples were quantified using real-time PCR.

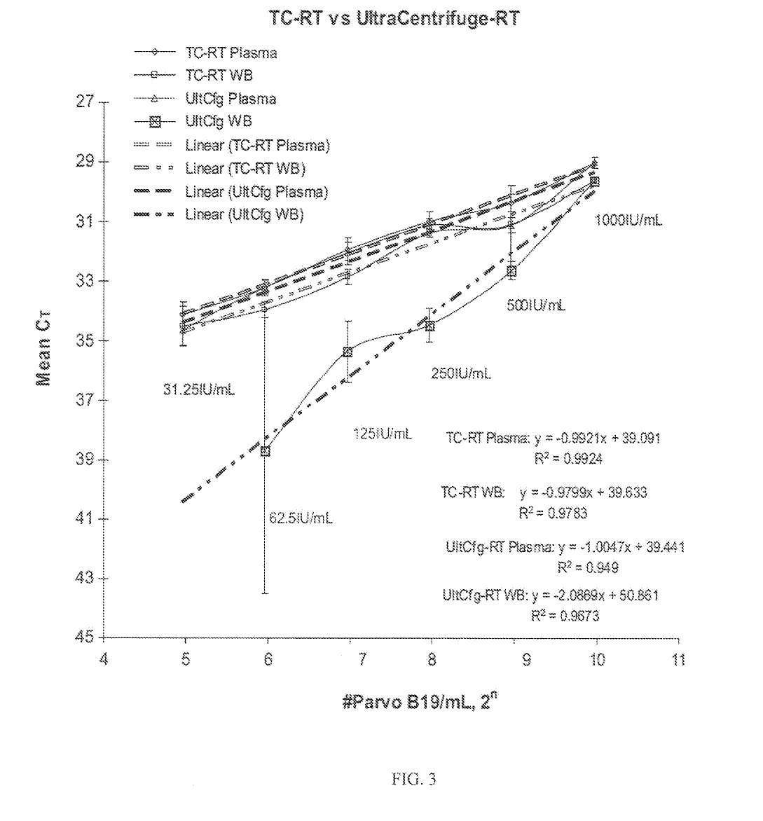

The results are shown in FIG. 3. The x-axis indicates viral input per milliliter in base 2 log format. The y-axis indicates the mean C_{T}. The error bars show ±1 standard deviation. A trendline was drawn through each of the four data sets and the linear equation for each was generated. The slope for TC-RT plasma was −0.9921, the slope for TC-RT WB was −0.9799, the slope for Ultracentrifuge-RT plasma was −1.004 and the slope for Ultracentrifuge-RT WB was −2.0869. The theoretical slope was −1.000.

The x-axis intercept of TC-RT plasma and TC-RT WB are 39.091 and 39.633, respectively. Given that the slopes of the 2 protocols are similar, this indicates that the difference between the 2 protocols is about 0.5 cycles.

Clinical Donor Specimen

The presence of Parvovirus B19 was analyzed in 111 paired donor plasma and whole blood samples. Table 3 shows the p-values between plasma and whole blood depending on the viral load levels of the sample. The viral load was broken down in 3 categories, 0−<10, 10−<100 and >100 copies/mL. As indicated, the recovery of virus is significantly higher in whole blood than in plasma.

TABLE 3

| | Viral Load, Mean +/− STD | | |
|---|---|---|---|
| Plasma Value | Plasma | Whole Blood | p value* |
| 0-<10 | 2.1 +/− 2.5 | 12.6 +/− 27.4 | 0.01 |
| 10-<100 | 35.2 +/− 20.4 | 71.8 +/− 102.6 | |
| >100 | 1.2E+05 +/− 5.3E+05 | 3E+06 +/− 1.6E+07 | |

*paired 2-tailed t-test

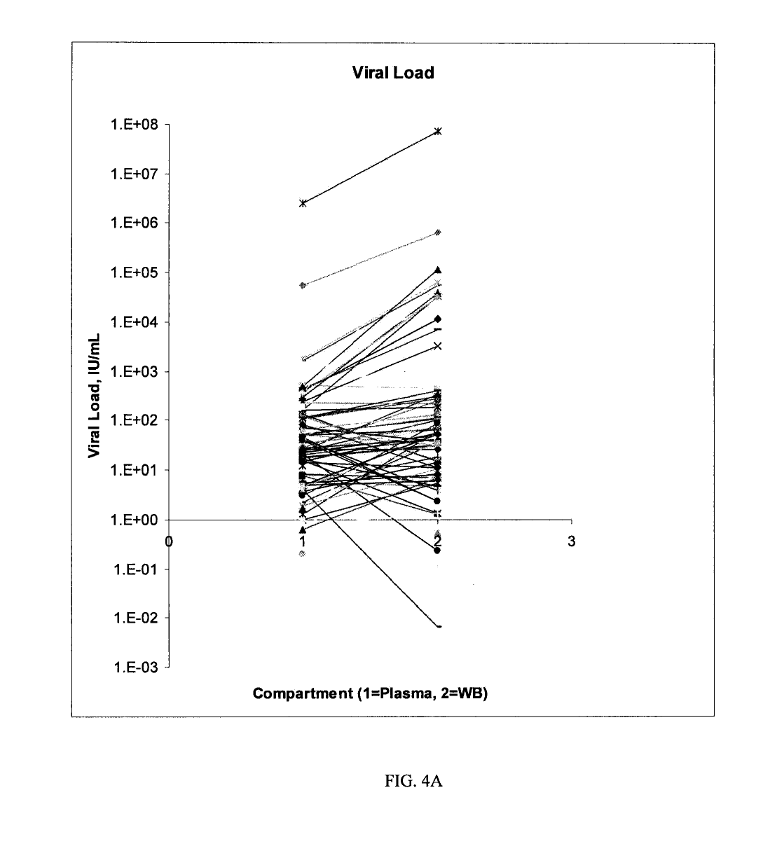

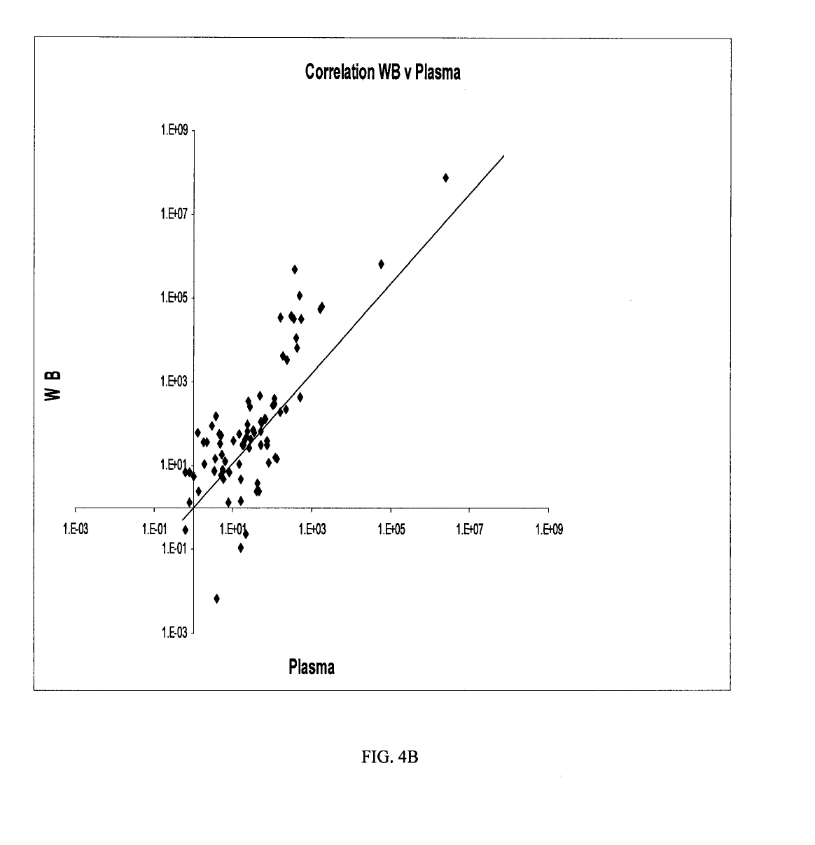

FIG. 4A is a graph depicting the viral load of Parvovirus B19 in clinical donor specimens using nucleic acid samples isolated from whole blood or plasma. FIG. 4B is a graph depicting the correlation of Parvovirus B19 nucleic acid recovery from whole blood verses plasma.

This example demonstrates that nucleic acids from Parvovirus B19 can be isolated from whole blood using the method of the invention.

Although the invention has been described with reference to the above examples, it will be understood that modifications and variations are encompassed within the spirit and scope of the invention. Accordingly, the invention is limited only by the following claims.

What is claimed is:

1. A method for detection of nucleic acid in a blood sample comprising:
a) treating the sample with a reagent that lyses cells;
b) contacting the sample with a salt based reagent to remove red blood cells (RBCs) and hemoglobin from the sample; and
c) detecting nucleic acid in the blood sample.

2. The method of claim 1, wherein reagent to remove RBCs and hemoglobin is a polyethylene glycol reagent.

3. The method of claim 2, wherein the polyethylene glycol is present in an amount of about 20 to 40% of the salt based reagent volume.

4. The method of claim 1, wherein the salt is a halide based salt.

5. The method of claim 1, wherein the salt is a chloride salt.

6. The method of claim 5, wherein the chloride salt is sodium chloride, potassium chloride, calcium chloride, ammonium chloride, or iron chloride.

7. The method of claim 1, wherein the salt is present in the salt based reagent in a concentration range of about 2.5-5.0 moles per liter.

8. The method of claim 1, further comprising after step b), centrifuging the sample to remove the RBCs and hemoglobin and lysed cells.

9. The method of claim 1, further comprising filtering the sample to remove the RBCs and hemoglobin and lysed cells.

10. The method of claim 1, further comprising settling the sample to remove the RBCs and hemoglobin.

11. The method of claim 1, further comprising detecting nucleic acid associated with infectious disease.

12. The method of claim 11, wherein the infectious disease is caused by a microorganism.

13. The method of claim 12, wherein the microorganism is a virus, a bacteria, or a parasite.

14. The method of claim 13, wherein the virus is selected from the group consisting of HIV, hepatitis A virus, HBV, HCV, WNV, Parvovirus B19, HTLV I/II, simian foamy virus, SARS, Dengue, ChikV, CMV, EBV, and HHV-8.

15. The method of claim 13, wherein the bacteria is selected from the group consisting of Escherichia, Proteus, Klebsiella, Staphylococcus, Streptococcus, Pseudomonas and Lactobacillus.

16. The method of claim 13, wherein the parasite is selected from the group consisting of Leishmania, Babesia, Treponema, Borrelia, Plasmodium and Trypanosoma.

17. The method of claim 1, wherein the reagent that lyses cells comprises guanidine-HCl.

18. A method for capturing a nucleic acid from a whole blood sample comprising:
a) lysing the cells in a sample;
b) adding a volume of a reagent comprising polyethylene glycol (PEG) and salt equal to the sample in a);
c) clarifying the sample to leave nucleic acid in the plasma; and
d) capturing the nucleic acid.

19. The method of claim 18, wherein the cells are lysed with a reagent comprising a guanidine-salt.

20. The method of claim 18, wherein the polyethylene glycol is present in an amount of about 20 to 40% of the reagent volume.

21. The method of claim 18, wherein the salt is a halide salt.

22. The method of claim 18, wherein the salt is a chloride salt.

23. The method of claim 5, wherein the chloride salt is sodium chloride, potassium chloride, calcium chloride, ammonium chloride, or iron chloride.

24. The method of claim 1, wherein the salt is present in the reagent in a concentration range of about 2.5-5.0 moles per liter.

25. The method of claim 18, wherein the nucleic acid is DNA or RNA.

26. The method of claim 25, wherein the nucleic acid is viral, prokaryotic, or eukaryotic in origin.

27. The method of claim 26, wherein the nucleic acid is derived from a virus selected from the group consisting of HIV, hepatitis A virus, HBV, HCV, WNV, Parvovirus B19, HTLV I/II, simian foamy virus, SARS, Dengue, ChikV, CMV, EBV, and HHV-8.

28. The method of claim 26, wherein the prokaryotic DNA is derived from a prokaryote selected from the group consisting of Escherichia, Proteus, Klebsiella, Staphylococcus, Streptococcus, Pseudomonas and Lactobacillus.

29. The method of claim 26, wherein the eukaryotic nucleic acid is derived from a eukaryote selected from the group consisting of algae, protozoa, parasites, fungi, molds and mammalian cells.

30. The method of claim 26, wherein the nucleic acid is derived from a parasite selected from the group consisting of Plasmodium, Borrelia, Leishmania, Babesia, Treponema and Trypanosoma.

31. A method of diagnosing a subject as having or at risk of having a transfusion transmitted infection (TTI), comprising detecting the presence of TTI nucleic acids in a sample from the subject, wherein detecting the presence of TTI nucleic acids is diagnostic of a TTI.

32. A kit for performing the method of claim 1 or 18, comprising polyethylene glycol (PEG), sodium chloride and guanidine HCl in one or more vials.

33. A method preparing a blood sample for nucleic acid analysis comprising:
a) contacting a blood sample with a lysis reagent to form a lysed blood sample;
b) contacting the lysed blood sample with a reagent that binds to blood cells and hemoglobin to form particles of reagent bound to the blood cells and hemoglobin; and
c) separating the particles from the supernatant, wherein the nucleic acids remain in the supernatant, thereby preparing the sample for nucleic acid analysis.

34. The method of claim 33, wherein the separating step includes settling, filtering, or centrifuging.

35. The method of claim 33, further comprising, isolating the supernatant.

36. The method of claim 33, wherein the lysis reagent comprises a chaotropic agent.

37. The method of claim 36, wherein the chaotropic agent is selected from the group consisting of guanidinium chloride, guanidine HCl, urea, and lithium perchlorate.

38. The method of claim 33, wherein the reagent that binds to blood cells and hemoglobin comprises polyethylene glycol.

39. The method of claim 38, wherein the polyethylene glycol is present in an amount of about 20 to about 40% of the reagent volume.

40. The method of claim 38, wherein the reagent comprises a salt.
